(19) United States
(12) Patent Application Publication
(10) Pub. No.: US 2024/0307104 A1
(43) Pub. Date: Sep. 19, 2024

(54) HIGH-FREQUENCY ELECTROSURGICAL DEVICE WITH AUTOMATIC POWER CUT-OFF FUNCTION

(71) Applicants: Jongho SONG, Seoul (KR); Siwoo PARK, Anyang-si (KR)

(72) Inventors: Jongho SONG, Seoul (KR); Siwoo PARK, Anyang-si (KR); Sechan PARK, Anyang-si (KR)

(21) Appl. No.: 18/603,619

(22) Filed: Mar. 13, 2024

(30) Foreign Application Priority Data
Mar. 13, 2023 (KR) 10-2023-0032843

(51) Int. Cl. A61B 18/12; A61B 18/00; H02H 7/20; H03K 3/017

(52) U.S. Cl. CPC A61B 18/1206; H02H 7/20; H03K 3/017; A61B 2018/00595; A61B 2018/00708; A61B 2018/00767; A61B 2018/00827; A61B 2018/00875; A61B 2018/00892; A61B 2018/1286

(57) ABSTRACT

A high-frequency electrosurgical device with an automatic power cut-off function, which may cauterize a tissue in an affected area by applying a high frequency to a surgical area of a patient, may include: a high frequency generator outputting the high frequency; a first electrode and a second electrode, each connected to the high frequency generator to receive the generated high frequency, and each having one end connected to a patient body to provide the high frequency; a detection unit disposed between the high frequency generator and at least one of the first electrode and the second electrode, and measuring at least one of the high frequency voltage and current applied thereto; and a control unit receiving an operation instruction and controlling the high frequency generator based on at least one of the voltage and the current, measured by the detection unit.

### CROSS-REFERENCE TO RELATED APPLICATIONS

This application claims priority under 35 U.S.C. § 119 to Korean Patent Application No. 10-2023-0032843, filed on Mar. 13, 2023, in the Korean Intellectual Property Office, the disclosure of which is incorporated herein by reference in its entirety.

### TECHNICAL FIELD

The following disclosure relates to a high-frequency electrosurgical device with an automatic power cut-off function, and more particularly, to a high-frequency electrosurgical device with an automatic power cut-off function which may cut off power for power not to be continuously supplied after the cut or cauterization of a patient tissue by measuring a resistance value of a corresponding area in real time to thus determine a cut or cauterization state while supplying power to a surgical area of a patient to thus cut or cauterize the tissue in the corresponding area.

### BACKGROUND

The statements in this section merely provide background information related to the present disclosure and may not constitute prior art.

A high frequency electrosurgical device may be a device which may incise, excise, or cauterize a patient tissue by using electrical energy generated from a high frequency current.

The high-frequency current may flow between two electrodes connected to an affected area of a patient. The tissues between these electrodes may have different resistance values depending on a thickness of the tissue, a composition (e.g., moisture or fat content) of the tissue, or the like, which may be equivalent to a plurality of resistors having various resistance values and connected in series with each other.

When the high-frequency current flows, according to a formula of P=I²R, the most severe heat may occur in a part having the highest resistance value among the tissues in a current path (a thinnest tissue if the tissues have compositions similar to each other) due to its resistance loss, which may lead to an increased temperature.

Accordingly, moisture density that contributes to current transmission may be gradually decreased due to the increased temperature, which makes the resistance value larger, and moisture evaporation may occur when the increased temperature reaches to a boiling point or more.

This moisture evaporation may cause a synergistic effect of rapidly increasing the resistance value, and as a result, the corresponding tissue may be burned to achieve a surgical purpose.

However, in such a conventional high-frequency electrosurgical device, an operator may cut off a power output of the device at a set time point by using a timer function installed in the surgical device, or cut off the power output by manipulating a switch at an appropriate time point based on the operator's experience.

Accordingly, in a surgical method using the conventional high-frequency electrosurgical device, the power output of the surgical device may be simply cut off based on the operator's experience in a state where it is impossible for the operator to accurately recognize whether the affected area to be cut is cut, thus causing a side effect of unintentional overexposure of the high frequency current to the patient.

This side effect may cause a variety of problems that may cause unwanted damage to a tissue surrounding the affected area or cause pain for the patient.

In addition, the power output used in a dental high-frequency electrosurgical device may use a high frequency and have a waveform similar to a square wave rather than a sine wave. To acquire an accurate effective output voltage of this waveform, the output voltage may be calculated using an analog to digital (A/D) conversion result of its high-speed sampling of one cycle or several cycles in synchronization with the output waveform, which may increase a device price.

Korean Publication No. 10-2006-0074949 A, published on Jul. 3, 2006, relates to an electrode for radiofrequency tissue ablation.

The above information disclosed in this background section is only for enhancement of understanding of the background of the present disclosure, and therefore it may contain information that does not form the prior art that is already known to a person of ordinary skill in the art.

### SUMMARY

An embodiment of the present invention is directed to providing a high-frequency electrosurgical device with an automatic power cut-off function which may cut off power for power not to be continuously supplied after the cut or cauterization of a patient tissue by measuring a resistance value of a corresponding area in real time to thus determine a cut or cauterization state while supplying power to a surgical area of a patient to thus cut or cauterize the tissue in the corresponding area.

The present invention may have the following features to solve the above problem.

In one general aspect, provided is a high-frequency electrosurgical device, which may cauterize a tissue in an affected area by applying a high frequency to a surgical area of a patient, the device including: a high frequency generator outputting the high frequency; a first electrode and a second electrode, each connected to the high frequency generator to receive the generated high frequency, and each having one end connected to a patient body to provide the high frequency; a detection unit disposed between the high frequency generator and at least one of the first electrode and the second electrode, and measuring at least one of the high frequency voltage and current applied thereto; and a control unit receiving an operation instruction and controlling the high frequency generator based on at least one of the voltage and the current, measured by the detection unit, wherein the control unit measures a resistance value of the surgical area of the patient in real time to determine a state of the affected area, and controls the high frequency generator to cut-off the high frequency output based on the state of the affected area.

The control unit may control output power of the high frequency generator for the high frequency generator to output the high frequency of a predetermined power amount, and determine that the tissue in the affected area is completely cauterized when a magnitude of the output voltage of the high frequency generator is greater than a predetermined reference, or a change amount of the output voltage is outside a predetermined range.

The control unit may stop an operation of the high frequency generator for the output of the high frequency generator to be cut off, and provide a cauterization completion state when determining that the tissue in the affected area is completely cauterized.

The control unit may include a general controller generating a power instruction, and a power controller controlling the high frequency generator for the high frequency generator to output predetermined power based on the power instruction.

The high frequency generator may include a buck converter connected to an input power source to convert a voltage of the input power source to a low voltage and lowering the output voltage for the output voltage to be output as a voltage of a desired magnitude under control of the power controller, a half-bridge converter connected to the buck converter and generating a pulse width modulation (PWM) voltage of the predetermined frequency and duty under control of the general controller, and a transformer having a first side connected to the half bridge converter and a second side insulated from the first side, and providing the output voltage to the first second electrodes connected to the second side by transforming the output voltage of the half bridge converter.

The detection unit may include at least one of a voltage detection unit connected to the other end of the first electrode and detecting the voltage output to the first electrode, and a current detection unit connected to the other end of the second electrode and detecting the current flowing into the second electrode.

The device may further include a shunt resistor connected between the second electrode and the transformer.

The voltage detection unit may include a first resistor having one end connected to the other end of the first electrode, a second resistor having one end connected to the other end of the first resistor, a V_REF voltage source connected to the other end of the second resistor, and a first amplifier connected to the other end of the first resistor.

The voltage detection unit may further include a first voltage comparator having a positive input terminal connected to an output terminal of the first amplifier and a negative input terminal connected to the V_REF voltage source, and comparing and outputting the voltage of the V_REF voltage source and the output voltage of the first amplifier.

The current detection unit may include a third resistor having one end connected to the other end of the second electrode, a fourth resistor having one end connected to the other end of the third resistor, a VCC voltage source connected to the other end of the fourth resistor, and a second amplifier connected to the other end of the third resistor and outputting the voltage when the high frequency current is zero due to divided voltages of the third resistor and the fourth resistor.

The current detection unit may further include a second voltage comparator having a positive input terminal connected to an output terminal of the second amplifier and a negative input terminal connected to the V_REF voltage source, and comparing and outputting the voltage of the V_REF voltage source and the output voltage of the second amplifier.

The V_REF voltage source may include a VCC voltage source, a fifth resistor having one end connected to the VCC voltage source, a sixth resistor having one end connected to the other end of the fifth resistor and the other end grounded, and a third amplifier connected to the other end of the fifth resistor, wherein the output voltage of the V_REF voltage source is the output voltage of the third amplifier, and the fifth resistor and the sixth resistor have the same resistance value.

The control unit may perform an analog to digital (A/D) conversion and sampling on an output voltage value of the first voltage comparator or the second voltage comparator, and perform the A/D conversion on the output voltage value after a predetermined first time elapses from a reception time point of the output voltage value when an interruption occurs at the output voltage value of the first voltage comparator or the second voltage comparator.

The control unit may perform scaling by multiplying the output voltage value by a constant k that is based on a desired scaling magnitude for the output voltage value to be close to an effective value when performing the sampling.

The control unit may perform the A/D conversion on the output voltage value of the V_REF voltage source based on a preset time interval, and determine a fault of the V_REF voltage source, stop the operation of the high frequency generator, and provide fault state information of the V_REF voltage source when the output voltage is more than a preset first reference voltage.

The control unit may receive the output voltage value after stopping the output of the high frequency generator when detecting that the output voltage value of the sampled first voltage comparator is a maximum value within a preset input range, and determine a fault of the voltage detection unit, stop the operation of the high frequency generator, and provide fault state information of the voltage detection unit when the received output voltage value is more than a preset second reference voltage.

The control unit may increase the duty of the buck converter when detecting that the output voltage value of the sampled first voltage comparator is a minimum value within a preset input range, and then determine a fault of the high frequency generator, stop the operation of the high frequency generator, and provide fault state information of the high frequency generator when the output voltage value of the first voltage comparator does not reach a preset expected voltage value.

The control unit may compute the output current flowing through the shunt resistor based on the output voltage value of the sampled second voltage comparator, stop the output of the high frequency generator and compute the output current when detecting that the output current is a maximum value within a preset input range, and determine a fault of the current detection unit, stop the operation of the high frequency generator, and provide fault state information of the current detection unit when the received output current is more than a preset first reference current.

The control unit may compute the output current flowing through the shunt resistor based on the output voltage value of the sampled second voltage comparator, increase the duty of the buck converter when detecting that the output current is a minimum value within a preset input range, and then determine a fault of the high frequency generator, stop the operation of the high frequency generator, and provide fault state information of the high frequency generator when the output current flowing through the shunt resistor does not reach a preset expected current value.

When controlling the duty of the buck converter, the control unit may compute a maximum duty for ensuring the magnitude of the voltage supplied to the surgical area of the patient not to be more than 610 V, and perform the control for the duty transmitted to the buck converter to be smaller than the maximum duty.

### BRIEF DESCRIPTION OF THE DRAWINGS

FIG. 1 is a control block diagram of a high-frequency electrosurgical device according to an embodiment of the present invention.

FIG. 2 is a circuit diagram showing a configuration of an output circuit part of the high-frequency electrosurgical device according to an embodiment of the present invention.

FIG. 3 is a schematic circuit diagram of a high frequency generator of FIG. 2.

FIG. 4 is a circuit diagram of a detection unit of FIG. 2.

FIGS. 5A to 5C are graphs each showing sampling performed by a control unit according to an embodiment of the present invention.

### DETAILED DESCRIPTION OF EMBODIMENTS

Hereinafter, embodiments of the present invention are described in detail with reference to the accompanying drawings so that those skilled in the art to which the present invention pertains may easily practice the present invention. The present invention may be modified in various different forms, and is not limited to the embodiments provided herein. A portion unrelated to the description is omitted in order to obviously describe the present invention, and the same or similar components are denoted by the same reference numeral throughout the specification. In addition, a detailed description of the widely and well known art is omitted.

Throughout the specification, unless explicitly described to the contrary, “including” any component should be understood to imply the inclusion of another element rather than the exclusion of another element.

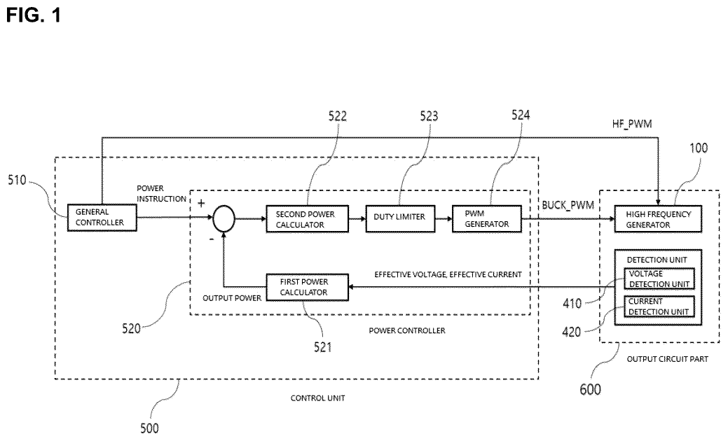

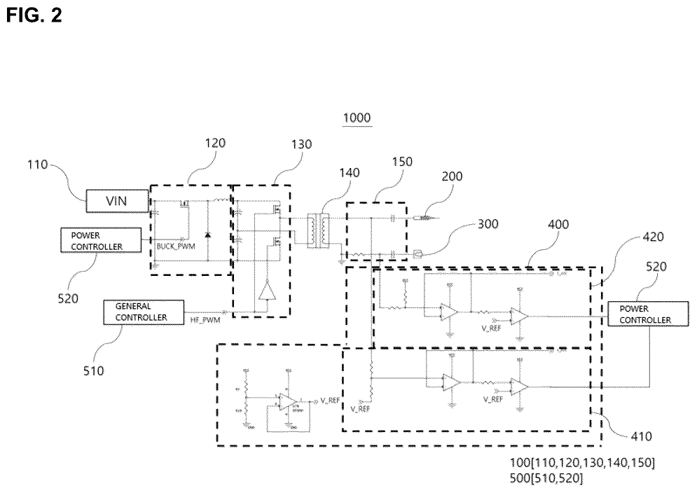

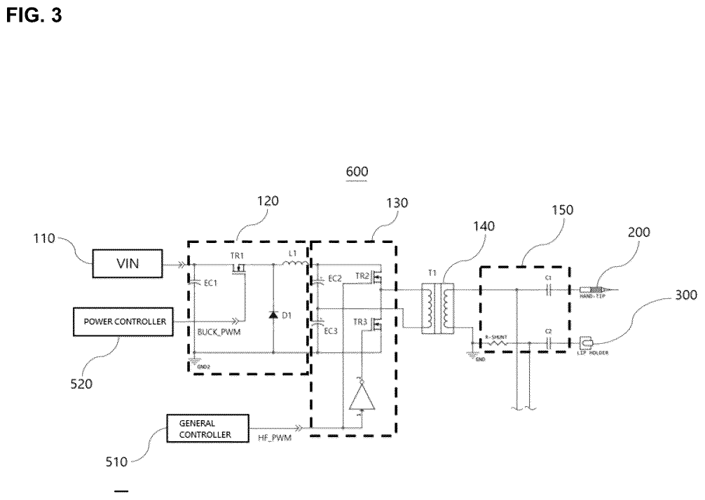

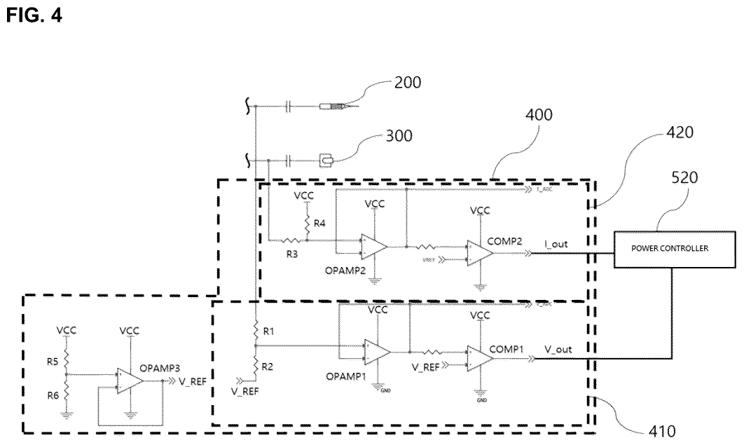

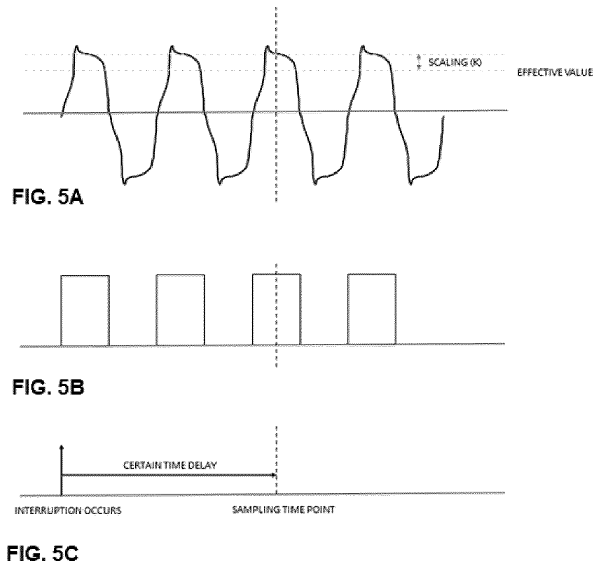

FIG. 1 is a control block diagram of a high-frequency electrosurgical device according to an embodiment of the present invention; FIG. 2 is a circuit diagram showing a configuration of an output circuit part of the high-frequency electrosurgical device according to an embodiment of the present invention; FIG. 3 is a schematic circuit diagram of a high frequency generator of FIG. 2; FIG. 4 is a circuit diagram of a detection unit of FIG. 2; and FIGS. 5A to 5C are graphs each showing sampling performed by a control unit according to an embodiment of the present invention.

Referring to the drawings, a high-frequency electrosurgical device 1000 according to an embodiment of the present invention may roughly include: a high frequency generator 100 outputting a high frequency; a first electrode 200 and a second electrode 300, each connected to the high frequency generator 100 to receive the generated high frequency, and each having one end connected to a patient body to provide the high frequency; a detection unit 400 disposed between the high frequency generator 100 and at least one of the first electrode 200 and the second electrode 300, and measuring at least one of the high frequency voltage and current applied thereto; and a control unit 500 receiving an operation instruction and controlling the high frequency generator 100 based on at least one of the voltage and the current, measured by the detection unit 400.

Here, the high frequency generator 100 may output the voltage and current of a desired high frequency based on pulse width modulation (PWM) control of the control unit 500. The high frequency generator 100 may include an input power source 110, a buck converter 120 connected to the input power source 110 to convert a voltage of the input power source 110 to a low voltage and outputting an output voltage at a desired duty under control of a power controller 520, a half-bridge converter 130 connected to the buck converter 120 and outputting the output voltage at the set frequency and duty under control of a general controller 510, a transformer 140 having a first side connected to the half bridge converter 130 and a second side insulated from the first side, and providing the output voltage to the first and second electrodes 200 and 300 connected to the second side by transforming the output voltage of the half bridge converter 130, and a filter unit 150 disposed at the front of the first and second electrodes 200 and 300 to filter noise input to the first and second electrodes 200 and 300.

Here, the buck converter 120 may be a converter connected to the input power source 110 to convert a high voltage to the low voltage, may be a step-down CHOPPER type converter, and may control output power of the high frequency generator 100 by receiving a PWM signal BUCK_PWM for receiving a duty instruction based on the PWM control of the power controller 520 described below and input to a gate terminal of TR1 in FIG. 3.

In addition, the half-bridge converter 130 may control the output power of the high frequency generator 100 at the set duty and the specific frequency set based on the PWM control of the general controller 510 described below.

The PWM signal controlled by the general controller 510 may be an HF_PWM signal and, in one example, the PWM signal may be output at a frequency of 510 kHz and a duty of 50%.

That is, the output voltage control of the high frequency generator 100 by the general controller 510 may be the preset basic frequency and duty, and the power controller 520 may receive the voltage and the current, measured in real-time, from the detection unit 400 described below, and output the BUCK_PWM signal to the buck converter to satisfy an initially received output power instruction based on a real-time resistance change in a surgical area of a patient, thereby controlling the output power provided to the first electrode and the second electrode.

Meanwhile, one end of each of the first electrode 200 and the second electrode 300 may be connected to the surgical area of the patient. In an embodiment of the present invention, the surgical area of the patient may be applied to a patient tooth, the first electrode 200 may be connected to a nerve of the tooth, and the second electrode 300 may be connected to patient lips.

The surgical area to which the first electrode 200 and the second electrode 300 of the present invention are connected may be applied to any area of the patient body. However, it is possible to ensure a higher cauterization performance as a body area to which the first electrode 200 is connected and a body area to which the second electrode 300 is connected are closer to each other.

Meanwhile, the detection unit 400 may be disposed between the high frequency generator 100 and at least one of the first electrode 200 and the second electrode 300 and measure at least one of the high frequency voltage and current applied thereto. The detection unit 400 may include a voltage detection unit 410 connected to the other end of the first electrode 200 and detecting the voltage output to the first electrode 200, and a current detection unit 420 connected to the other end of the second electrode 300 and detecting the current flowing into the second electrode 300.

Here, the voltage detection unit 410 may include a first resistor R1 having one end connected to the other end of the first electrode 200, a second resistor R2 having one end connected to the other end of the first resistor R1, a V_REF voltage source V_REF connected to the other end of the second resistor R2, a first amplifier OPAMP1 connected to the other end of the first resistor R1, and a first voltage comparator COMP1 having a positive input terminal connected to an output terminal of the first amplifier OPAMP1 and a negative input terminal connected to the V_REF voltage source V_REF, and comparing and outputting the voltage of the V_REF voltage source and the output voltage of the first amplifier OPAMP1.

Accordingly, the output voltage supplied to the other end of the first electrode 200 may be divided into the low voltage by the first resistor and the second resistor, amplified by the first amplifier, and then detected by the first voltage comparator. An output voltage value may thus be provided to the power controller 520 of the control unit 500.

In addition, the current detection unit 420 may include a third resistor R3 having one end connected to the other end of the second electrode 300, a fourth resistor R4 having one end connected to the other end of the third resistor R3, a VCC voltage source VCC connected to the other end of the fourth resistor R4, a second amplifier OPAMP2 connected to the other end of the third resistor R3 and outputting the voltage when the high frequency current is zero due to divided voltages of the third resistor R3 and the fourth resistor R4, and a second voltage comparator COMP2 having a positive input terminal connected to an output terminal of the second amplifier and a negative input terminal connected to the V_REF voltage source V_REF, and comparing and outputting the voltage of the V_REF voltage source and the output voltage of the second amplifier OPAMP2.

As shown in FIG. 2, in the current detection unit 420, the current flowing in the other end of the second electrode 300 may be the current transmitted to the second side of the transformer 140, and flow through a current movement path passing through C1, the first electrode 200, the second electrode 300, C2, and a shunt resistor R_SHUNT.

Here, an amount I of the flowing current may be expressed as a magnitude of a voltage V=IR_SHUNT applied across the shunt resistor R_SHUNT. Accordingly, the voltage proportional to the amount of the flowing current may appear at the output of the second amplifier OPAMP2 while having a magnitude of VCC/2 when the high frequency current becomes zero due to the divided pressures of the third resistor R3 and the fourth resistor R4.

In addition, the voltage and the current, passing through the first amplifier OPAMP1 and the second amplifier OPAMP2 may be input to the positive input terminal of each of the first voltage comparator (COMP1) and the second voltage comparator (COMP2), and the V_REF voltage source V_REF may be connected to the negative input terminal of each of the first voltage comparator COMP1 and the second voltage comparator (COMP2). Accordingly, when the output is generated from the transformer 140, a square wave may appear in each of the first voltage comparator COMP1 and the second voltage comparator COMP2.

Here, the V_REF voltage source V_REF may include a VCC voltage source VCC, a fifth resistor R5 having one end connected to the VCC voltage source VCC, a sixth resistor R6 having one end connected to the other end of the fifth resistor R5 and the other end grounded, and a third amplifier OPAMP3 connected to the other end of the fifth resistor R5.

Accordingly, the output voltage of the V_REF voltage source may be the output voltage of the third amplifier OPAMP3, and the fifth resistor R5 and the sixth resistor R6 may have the same resistance value. Accordingly, the V_REF voltage source may be ½ of the voltage of the VCC, and the V_REF voltage source may always maintain VCC/2 even when the VCC voltage source is not uniform.

Meanwhile, the control unit 500 may control the high frequency generator 100 based on at least one of the voltage and the current, measured by the detection unit 400. The control unit 500 may measure the resistance value of the surgical area of the patient in real time to determine a state of an affected area, and control the high frequency generator to cut-off the high frequency output based on the state of the affected area.

The control unit 500 may control the high frequency generator 100 to output a predetermined power amount, and determine that a tissue in the affected area is completely cauterized when a magnitude of the output voltage of the high frequency generator 100 is greater than a predetermined reference, or a change amount of the output voltage is outside a predetermined range.

In addition, the control unit 500 may stop an operation of the high frequency generator 100 and provide a cauterization completion state to the outside through the separate notification means or output means when determining that the tissue in the affected area is completely cauterized.

The control unit 500 described above may include the general controller 510 generating the power instruction, and the power controller 520 controlling the high frequency generator 100 for the high frequency generator 100 to output predetermined power based on the power instruction.

In addition, as shown in FIG. 1, the power controller 520 may include a first power calculator 521 calculating the output power currently being output from the high frequency generator 100 based on the current and the voltage, measured by the detection unit 400, a second power calculator 522 calculating power to be output based on the power instruction of the general controller 510 and the output power of the first power calculator 521, a duty limiter 523 checking whether the power to be output that is received from the second power calculator 522 corresponds to a duty limit, and a PWM generator 524 generating the PWM signal to have a duty corresponding to the power to be output.

Accordingly, the PWM signal generated by the PWM generator 524 may be input to the gate terminal of TR1 of the buck converter 120 to thus control the voltage at the second side of the transformer 140 to be changed.

Meanwhile, the control unit 500 may perform the analog to digital (A/D) conversion and the sampling on the output voltage value transmitted from the detection unit 400 to the power controller 520. A frequency of the output power used in the electrosurgical devices may be generally 510 KHz. Therefore, there is a need for a control unit capable of performing high-speed sampling to calculate the effective voltage and current of the high frequency voltage and current.

Here, the detection unit 400 may be provided with the first voltage comparator and the second voltage comparator to implement the control unit by using a low-end processor.

However, the output of the first or second voltage comparator may cause an (external) interrupt to the control unit at the rising edge or falling edge of the high frequency voltage and current. If this (external) interrupt occurs, the control unit 500 may perform the A/D conversion on the output voltage value after a predetermined first time elapses from a reception time point of the output voltage value of the first voltage comparator COMP1 or the second voltage comparator COMP2.

In addition, as shown in FIGS. 5A to 5C, the control unit 500 may preferably perform scaling by multiplying the output voltage value by a constant k that is based on a desired scaling magnitude for the output voltage value to be close to an effective value when performing the sampling. FIG. 5A shows an output voltage waveform or an output current waveform (here, the output voltage is taken as an example for convenience of explanation), and FIG. 5B shows an output waveform of the comparator disposed between the output voltage (or an output current) and ground, and shows that the output waveform is high if the output voltage (or the output current) is zero or more and the output waveform is low if the output voltage (or the output current) is less than zero. FIG. 5C shows that the external interrupt occurs at the rising edge of output of the comparator in FIG. 5B, the external interrupt is first disabled to prevent any further external interrupt occurrence when the external interrupt occurs, and the output voltage waveform is sampled after a certain time delay T_delay due to interruption of an internal timer. Here, a cycle of the output voltage waveform (or the output current waveform) may be referred to as T_out, and in this case, the delay time T_delay may preferably be set as in Equation 1 below. Here, n is an integer, and may be set by considering a calculation speed or the like of a processor being used. For example, when using a processor having a slow calculation speed, it is desirable to set n to a relatively large value to lengthen a sampling cycle.

$$T \_delay = n T \_Vout + m T \_out \qquad ( Equation 1 )$$

Here, m=(¼) or (¾). m indicates a time point at which the output voltage (or output current) waveform becomes flat. FIG. 5A shows an embodiment when m=(¼). A signal processing that is more robust to noise is possible by setting m in this way. The reason is that if m is set to be different from (¼) or (¾), the signal may be sampled at a time point that is not flat and has a steep slope, and may be greatly distorted even with small noise.

A sampled value of the output voltage (or the output current) acquired in this way may be multiplied by the preset constant k, thereby estimating the effective value of the output voltage (or the output current). The output used in a dental high-frequency electrosurgical device may use the high frequency of 510 KHz and have a waveform similar to the square wave rather than a sine wave. In order to perceive an accurate effective output voltage (or output current) of this waveform, the output voltage may be calculated by an A/D conversion result of the high-speed sampling of one cycle or several cycles in synchronization with the output waveform, which may be a factor of increasing a device price. On the other hand, the present invention may solve the above problem by estimating the effective value by multiplying the sampled value of the output voltage by k as above, thereby relatively lowering the device price.

In more detail, the interruption may occur by digitizing an output waveform signal, and thus occur in synchronization with the high frequency output waveform, and the certain time delay may also be caused by the interruption of the timer in the processor. Therefore, the sampling for the AD conversion may always be performed at the same time point within one cycle of the output waveform. A method for determining the constant k to convert this value to the effective value is as follows. First, an oscilloscope capable of high-speed calculation may be connected to a high frequency output terminal by using a high-voltage probe, and an effective value calculation function may then be turned on. Here, the constant k may be determined to ensure the effective value of the output waveform that is calculated by the oscilloscope and an AD conversion value to be the same as each other, and this value may be stored and used in a non-volatile memory.

Meanwhile, the high-frequency electrosurgical device 1000 may be a device directly connected to the body tissue, and its stability may thus be very important. Therefore, the device 100 needs to be configured to cut-off the output and generate an alarm to notify a user and protect the patient if an abnormal state occurs due to a circuit fault, or the like.

A fault area of the output circuit part 600 including the high frequency generator 100 and the detection unit 400 may roughly include a fault of the V_REF voltage source V_REF, faults of the voltage detection unit 410 and the current detection unit 420, and a fault of the high frequency generator 100.

Accordingly, the control unit 500 according to an embodiment of the present invention may perform the A/D conversion on the output voltage value of the V_REF voltage source V_REF based on a preset time interval, and determine the fault of the V_REF voltage source V_REF, stop the operation of the high frequency generator 100, and provide fault state information of the V_REF voltage source when the output voltage is more than a preset first reference voltage.

In addition, the control unit 500 may receive the output voltage value after stopping the output of the high frequency generator 100 when detecting that the output voltage value of the sampled first voltage comparator COMP1 is the maximum value within a preset input range, and determine the fault of the voltage detection unit 410, stop the operation of the high frequency generator 100, and provide fault state information of the voltage detection unit 410 when the received output voltage value is more than a preset second reference voltage.

In addition, the control unit 500 may increase the duty of the buck converter 120 when detecting that the output voltage value of the sampled first voltage comparator COMP1 is the minimum value within the preset input range, and then determine the fault of the high frequency generator 100, stop the operation of the high frequency generator 100, and provide fault state information of the high frequency generator 100 when the output voltage value of the first voltage comparator COMP1 does not reach a preset expected voltage value.

In addition, the control unit 500 may compute the output current flowing through the shunt resistor R_SHUNT based on the output voltage value of the sampled second voltage comparator COMP2, may stop the output of the high frequency generator 100 and compute the output current when detecting that the output current is the maximum value within the preset input range, and determine the fault of the current detection unit 420, stop the operation of the high frequency generator 100, and provide fault state information of the current detection unit 420 when the received output current is more than a preset first reference current.

In addition, the control unit 500 may compute the output current flowing through the shunt resistor R_SHUNT based on the output voltage value of the sampled second voltage comparator COMP2, may increase the duty of the buck converter 120 when detecting that the output current is the minimum value within the preset input range, and then determine the fault of the high frequency generator 100, stop the operation of the high frequency generator 100, and provide the fault state information of the high frequency generator 100 when the output current flowing through the shunt resistor R_SHUNT does not reach a preset expected current value.

Meanwhile, as in the duty limiter 523 described above, when controlling the duty of the buck converter 120, the control unit 500 may also preferably compute the maximum duty for ensuring a magnitude of the voltage supplied to the surgical area of the patient not to be more than 610 V, and perform the control for the duty transmitted to the buck converter 120 to be smaller than the maximum duty.

As set forth above, the high-frequency electrosurgical device of the present invention may cut off power for power not to be continuously supplied after the cut or cauterization of the patient tissue by measuring the resistance value of the corresponding area in real time to thus determine the cut or cauterization state when supplying power to the surgical area of the patient to thus cut or cauterize the tissue in the corresponding area, thereby preventing the damage to the patient body tissue other than the intended surgical area.

Although the embodiments are described in the present invention as above, the present invention is not limited to the specific embodiments described above. That is, it is apparent to those skilled in the art to which the present invention pertains that various variations and modifications could be made without departing from the spirit and scope of the appended claims, and all such appropriate variations and modifications should be considered as falling within the scope of the present invention as equivalents.

What is claimed is:

1. A high-frequency electrosurgical device which may cauterize a tissue in an affected area by applying a high frequency to a surgical area of a patient, the device comprising:
a high frequency generator outputting the high frequency;
a first electrode and a second electrode, each connected to the high frequency generator to receive the generated high frequency, and each having one end connected to a patient body to provide the high frequency;
a detection unit disposed between the high frequency generator and at least one of the first electrode and the second electrode, and measuring at least one of the high frequency voltage and current applied thereto; and
a control unit receiving an operation instruction and controlling the high frequency generator based on at least one of the voltage and the current, measured by the detection unit,
wherein the control unit measures a resistance value of the surgical area of the patient in real time to determine a state of the affected area, and controls the high frequency generator based on the state of the affected area.

2. The device of claim 1, wherein the control unit controls output power of the high frequency generator for the high frequency generator to output the high frequency of a predetermined power amount, and
determines that the tissue in the affected area is completely cauterized when a magnitude of the output voltage of the high frequency generator is greater than a predetermined reference, or a change amount of the output voltage is outside a predetermined range.

3. The device of claim 2, wherein the control unit stops an operation of the high frequency generator for the output of the high frequency generator to be cut off, and provides a cauterization completion state when determining that the tissue in the affected area is completely cauterized.

4. The device of claim 3, wherein the control unit includes
a general controller generating a power instruction, and
a power controller controlling the high frequency generator for the high frequency generator to output predetermined power based on the power instruction.

5. The device of claim 4, wherein the high frequency generator includes
a buck converter connected to an input power source to convert a voltage of the input power source to a low voltage and lowering the output voltage for the output voltage to be output as a voltage of a desired magnitude under control of the power controller,
a half-bridge converter connected to the buck converter and generating a pulse width modulation (PWM) voltage of the predetermined frequency and duty under control of the general controller, and
a transformer having a first side connected to the half bridge converter and a second side insulated from the first side, and providing the output voltage to the first second electrodes connected to the second side by transforming the output voltage of the half bridge converter.

6. The device of claim 5, wherein the detection unit includes at least one of
a voltage detection unit connected to the other end of the first electrode and detecting the voltage output to the first electrode, and
a current detection unit connected to the other end of the second electrode and detecting the current flowing into the second electrode.

7. The device of claim 6, further comprising a shunt resistor connected between the second electrode and the transformer.

8. The device of claim 7, wherein the voltage detection unit includes
a first resistor having one end connected to the other end of the first electrode,
a second resistor having one end connected to the other end of the first resistor,
a V_REF voltage source connected to the other end of the second resistor,
a first amplifier connected to the other end of the first resistor, and
a first voltage comparator having a positive input terminal connected to an output terminal of the first amplifier and a negative input terminal connected to the V_REF voltage source, and comparing and outputting the voltage of the V_REF voltage source and the output voltage of the first amplifier.

9. The device of claim 7, wherein the current detection unit includes
a third resistor having one end connected to the other end of the second electrode,
a fourth resistor having one end connected to the other end of the third resistor,
a VCC voltage source connected to the other end of the fourth resistor,
a second amplifier connected to the other end of the third resistor and outputting the voltage when the high frequency current is zero due to divided voltages of the third resistor and the fourth resistor, and
a second voltage comparator having a positive input terminal connected to an output terminal of the second amplifier and a negative input terminal connected to the V_REF voltage source, and comparing and outputting the voltage of the V_REF voltage source and the output voltage of the second amplifier.

10. The device of claim 8, wherein the V_REF voltage source includes
a VCC voltage source,
a fifth resistor having one end connected to the VCC voltage source,
a sixth resistor having one end connected to the other end of the fifth resistor and the other end grounded, and
a third amplifier connected to the other end of the fifth resistor,
wherein the output voltage of the V_REF voltage source is the output voltage of the third amplifier, and the fifth resistor and the sixth resistor have the same resistance value.

11. The device of claim 8, wherein the control unit performs an analog to digital (A/D) conversion and sampling on an output voltage value of the first voltage comparator or the second voltage comparator, and
performs the A/D conversion on the output voltage value after a predetermined first time elapses from a reception time point of the output voltage value when an interruption occurs at the output voltage value of the first voltage comparator or the second voltage comparator.

12. The device of claim 11, wherein the control unit performs scaling by multiplying the output voltage value by a constant k that is based on a desired scaling magnitude for the output voltage value to be close to an effective value when performing the sampling.

13. The device of claim 12, wherein the control unit performs the A/D conversion on the output voltage value of the V_REF voltage source based on a preset time interval, and
determines a fault of the V_REF voltage source, stops the operation of the high frequency generator, and provides fault state information of the V_REF voltage source when the output voltage is more than a preset first reference voltage.

14. The device of claim 12, wherein the control unit receives the output voltage value after stopping the output of the high frequency generator when detecting that the output voltage value of the sampled first voltage comparator is a maximum value within a preset input range, and
determines a fault of the voltage detection unit, stops the operation of the high frequency generator, and provides fault state information of the voltage detection unit when the received output voltage value is more than a preset second reference voltage.

15. The device of claim 12, wherein the control unit increases the duty of the buck converter when detecting that the output voltage value of the sampled first voltage comparator is a minimum value within a preset input range, and
then determines a fault of the high frequency generator, stops the operation of the high frequency generator, and provides fault state information of the high frequency generator when the output voltage value of the first voltage comparator does not reach a preset expected voltage value.

16. The device of claim 12, wherein the control unit computes the output current flowing through the shunt resistor based on the output voltage value of the sampled second voltage comparator,
stops the output of the high frequency generator and computes the output current when detecting that the output current is a maximum value within a preset input range, and
determines a fault of the current detection unit, stops the operation of the high frequency generator, and provides fault state information of the current detection unit when the received output current is more than a preset first reference current.

17. The device of claim 12, wherein the control unit computes the output current flowing through the shunt resistor based on the output voltage value of the sampled second voltage comparator,
increases the duty of the buck converter when detecting that the output current is a minimum value within a preset input range, and
then determines a fault of the high frequency generator, stops the operation of the high frequency generator, and provides fault state information of the high frequency generator when the output current flowing through the shunt resistor does not reach a preset expected current value.

18. The device of claim 12, wherein when controlling the duty of the buck converter, the control unit computes a maximum duty for ensuring the magnitude of the voltage supplied to the surgical area of the patient not to be more than 610 V, and
performs the control for the duty transmitted to the buck converter to be smaller than the maximum duty.
